SEQUENCE LISTING (22 sequences)

SEQ ID NO: 1 - protein, 56 aa, Staphylococcus aureus
AQHDEAQQNAFYQVLNMPNLNADQRNGFIQSLKDDPSQSANVLGEAQKLNDSQAPK

SEQ ID NO: 2 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 3 - protein, 58 aa, Staphylococcus aureus
ADNNFNKEQQNAFYEILNMPNLNEEQRNGFIQSLKDDPSQSANLLAEAKKLNESQAPK

SEQ ID NO: 4 - protein, 58 aa, Staphylococcus aureus [sequence omitted]

SEQ ID NO: 5 - protein, 58 aa, Staphylococcus aureus
ADNKFNKEQQNAFYEILHLPNLTEEQRNGFIQSLKDDPSVSKEILAEAKKLNDAQAPK

SEQ ID NO: 6 - protein, 116 aa, Artificial Sequence
  C domain mutant
ADNRFNREQQNAFYEILHLPNLTEEQRNAFIQRLRDDPSVSREILAEAQRLNDAQAPKADNRFNREQQNAFYEILHLPNLTEEQRNAFIQRLRDDPSVSREILAEAQRLNDAQAPK

SEQ ID NO: 7 - protein, 116 aa, Artificial Sequence
  C domain mutant
ADNQFNREQQNAFYEILHLPNLTEEQRNAFIQRLRDDPSVSREILAEAQRLNDAQAPKADNQFNREQQNAFYEILHLPNLTEEQRNAFIQRLRDDPSVSREILAEAQRLNDAQAPK

SEQ ID NO: 8 - DNA, 359 bp, Artificial Sequence
  synthetic DNA
ctgcagataaccgttttaaccgtgaacaacaaaacgctttctacgaaatcctgcacttgccaaaccttactgaagaacaacgtaatgctttcatccaacgtctgcgcgatgatccatctgtatcccgtgaaattttggcagaggctcaacgacttaacgacgctcaggcgcctaaggcagataaccgttttaaccgtgaacaacaaaacgctttctacgaaatcctgcacttgccaaaccttactgaagaacaacgtaatgctttcatccaacgtctgcgcgatgatccatctgtatcccgtgaaattttggcagaggctcaacgacttaacgacgctcaggcgcctaagtaatctaga

SEQ ID NO: 9 - DNA, 359 bp, Artificial Sequence
  synthetic DNA
ctgcagataaccaatttaaccgtgaacaacaaaacgctttctacgaaatcctgcacttgccaaaccttactgaagaacaacgtaatgctttcatccaacgtctgcgcgatgatccatctgtatcccgtgaaattttggcagaggctcaacgacttaacgacgctcaggcgcctaaggcagataaccaatttaaccgtgaacaacaaaacgctttctacgaaatcctgcacttgccaaaccttactgaagaacaacgtaatgctttcatccaacgtctgcgcgatgatccatctgtatcccgtgaaattttggcagaggctcaacgacttaacgacgctcaggcgcctaagtaatctaga

SEQ ID NO: 10 - protein, 290 aa, Artificial Sequence
  C domain mutant
ADNRFNREQQNAFYEILHLPNLTEEQRNAFIQRLRDDPSVSREILAEAQRLNDAQAPKADNRFNREQQNAFYEILHLPNLTEEQRNAFIQRLRDDPSVSREILAEAQRLNDAQAPKADNRFNREQQNAFYEILHLPNLTEEQRNAFIQRLRDDPSVSREILAEAQRLNDAQAPKADNRFNREQQNAFYEILHLPNLTEEQRNAFIQRLRDDPSVSREILAEAQRLNDAQAPKADNRFNREQQNAFYEILHLPNLTEEQRNAFIQRLRDDPSVSREILAEAQRLNDAQAPK

SEQ ID NO: 11 - DNA, 881 bp, Artificial Sequence
  synthetic DNA
ctgcagataaccgttttaaccgtgaacaacaaaacgctttctacgaaatcctgcacttgccaaaccttactgaagaacaacgtaatgctttcatccaacgtctgcgcgatgatccatctgtatcccgtgaaattttggcagaggctcaacgacttaacgacgctcaggcgcctaaggctgataaccgcttcaaccgagaacaacaaaacgctttttatgaaatccttcacctgccaaatcttacagaagaacaacgcaacgcattcattcaacgcttgcgtgatgacccttccgttagccgagagatcctggctgaagcacaacgtttgaatgatgcgcaagcaccaaaagctgataatcgattcaaccgtgaacaacaaaatgcattctacgaaatcttgcaccttcctaacctgactgaagagcagcgtaacgcttttatccagcgtttgagagacgatccatctgtctcccgtgaaattctcgcagaagcgcaacgcctgaacgatgctcaagctccgaaagcagacaaccgtttcaatcgcgaacagcaaaacgcgttttatgaaattctgcatcttccaaacttgacagaggaacaacgcaatgctttcatccaacgactgcgtgatgatccgagcgtttctcgagaaatcttggctgaagcacaacgtctgaacgacgctcaagctccaaaagcggataacagatttaaccgtgaacaacaaaatgctttctacgagatcttgcaccttccgaacctgactgaagaacaacgtaacgcatttattcagagattgcgcgatgacccatccgtaagccgtgagatcctggcagaagctcaacgtttgaatgatgcacaagctccaaaataatctaga

SEQ ID NO: 12 - DNA, 30 bp, Artificial Sequence
  primer 1
ttcgctgcagataaccaatttaaccgtgaa

SEQ ID NO: 13 - DNA, 30 bp, Artificial Sequence
  primer 2
actatctagattattttggagcttgtgcat

SEQ ID NO: 14 - protein, 116 aa, Artificial Sequence
  C-G29A.2d
ADNKFNKEQQNAFYEILHLPNLTEEQRNAFIQSLKDDPSVSKEILAEAKKLNDAQAPKADNKFNKEQQNAFYEILHLPNLTEEQRNAFIQSLKDDPSVSKEILAEAKKLNDAQAPK

SEQ ID NO: 15 - DNA, 351 bp, Artificial Sequence
  C-G29A.2d
gcagataacaaatttaacaaagaacaacaaaacgctttctacgaaatcctgcacttgccaaaccttactgaagaacaacgtaatgctttcatccaatccctgaaagatgatccatctgtatccaaagaaattttggcagaggctaaaaaacttaacgacgctcaggcgcctaaggctgataacaaattcaataaagaacagcaaaacgctttttatgaaatccttcacctgccaaatcttacagaagaacaacgcaacgcattcattcaaagcttgaaggatgacccttccgttagcaaagagatcctggctgaagcaaaaaagttgaatgatgcgcaagcaccaaaataa

SEQ ID NO: 16 - protein, 115 aa, Artificial Sequence
  C-G29A.2d-K07T
ADNKFNTQQNAFYEILHLPNLTEEQRNAFIQSLKDDPSVSKEILAEAKKLNDAQAPKADNKFNKEQQNAFYEILHLPNLTEEQRNAFIQSLKDDPSVSKEILAEAKKLNDAQAPK

SEQ ID NO: 17 - DNA, 351 bp, Artificial Sequence
  C-G29A.2d-K07T
gcagataacaaatttaacactgaacaacaaaacgctttctacgaaatcctgcacttgccaaaccttactgaagaacaacgtaatgctttcatccaatccctgaaagatgatccatctgtatccaaagaaattttggcagaggctaaaaaacttaacgacgctcaggcgcctaaggctgataacaaattcaataaagaacagcaaaacgctttttatgaaatccttcacctgccaaatcttacagaagaacaacgcaacgcattcattcaaagcttgaaggatgacccttccgttagcaaagagatcctggctgaagcaaaaaagttgaatgatgcgcaagcaccaaaataa

SEQ ID NO: 18 - DNA, 35 bp, Artificial Sequence
  Primer 3
ttcgcatatggcagataaccgttttaaccgtgaac

SEQ ID NO: 19 - DNA, 35 bp, Artificial Sequence
  Primer 4
ttttctgcagttattattttggagcttgtgcatca

SEQ ID NO: 20 - DNA, 35 bp, Artificial Sequence
  Primer 5
ttcgcatatggcagataaccaatttaaccgtgaac

SEQ ID NO: 21 - DNA, 35 bp, Artificial Sequence
  Primer 6
ttcgcatatggcagataaccgttttaacactgaac

SEQ ID NO: 22 - DNA, 35 bp, Artificial Sequence
  Primer 7
ttcgcatatggcagataaccaatttaacactgaac

(19) United States
(12) Patent Application Publication
(10) Pub. No.: US 2018/0170973 A1
(43) Pub. Date: Jun. 21, 2018

(54) IMMUNOGLOBULIN-BINDING MODIFIED PROTEIN

(71) Applicant: Kaneka Corporation, Osaka (JP)

(72) Inventors: Masakatsu Nishihachijyo, Hyogo (JP); Keita Yamashita, Osaka (JP); Osamu Odawara, Hyogo (JP); Takeyuki Tsuchidate, Hyogo (JP); Masayuki Takano, Hyogo (JP); Shinichi Yoshida, Hyogo (JP)

(73) Assignee: Kaneka Corporation, Osaka (JP)

(21) Appl. No.: 15/887,381

(22) Filed: Feb. 2, 2018

Related U.S. Application Data
(63) Continuation of application No. PCT/JP2016/072649, filed on Aug. 2, 2016.

(30) Foreign Application Priority Data
Aug. 4, 2015 (JP) 2015-154365

(51) Int. Cl. C07K 14/31; C07K 1/22; B01J 20/24; B01J 20/286; B01J 20/32

(52) U.S. Cl. CPC C07K 14/31; C07K 1/22; B01J 20/24; B01J 2220/52; B01J 20/3217; B01J 20/3278; B01J 20/286

(57) ABSTRACT

A protein includes two or more amino acid sequences, wherein each amino acid sequence is derived from a sequence selected from the group consisting of SEQ ID NOs:1 to 5. The amino acid sequence closest to the N-terminus includes a substitution of an amino acid residue at a position corresponding to position 4 or 7 of SEQ ID NO: 5 with an amino acid residue other than Arg, and productivity of the protein in a transformant producing the protein is improved, as compared to productivity of a protein including the amino acid sequence without the substitution.

### TECHNICAL FIELD

One or more embodiments of the present invention relate to a protein specifically binding to a target substance, a ligand affinity separation matrix on which the protein is immobilized, and a separation purification method using the matrix.

### BACKGROUND

The antibody drugs developed so far are mainly monoclonal antibodies, which are massively produced by, for example, recombinant cell culture techniques. The “monoclonal antibodies” refer to antibodies that are produced by clones of a single antibody-producing cell. Monoclonal antibodies produced by cultured cells are purified by a variety of chromatographic techniques to prepare drugs. Among chromatographic processes, affinity chromatographic processes, particularly using Protein A affinity separation matrices, provide one-step, high-purity purification of antibodies from animal cell cultures, and thus are essential in the preparation of antibody drugs.

Protein A is a cell wall protein produced by the gram-positive bacterium Staphylococcus aureus and contains a signal sequence S, five immunoglobulin-binding domains (E domain, D domain, A domain, B domain, and C domain), and a cell wall-anchoring domain known as XM region (Non-Patent Literature 1).

Protein A affinity separation matrices are prepared by immobilizing Protein A onto separation matrices. Protein A affinity separation matrices are generally expensive products because, for one reason, Protein A itself is a recombinant protein that is produced by a cultured transformant containing a foreign gene and then highly purified. Techniques that allow us to produce Protein A with high culture productivity are industrially valuable and thus there is a need to develop them.

Many techniques have been developed for highly functionalizing Protein A by modifying it through protein engineering. Examples of Protein A engineering techniques include those for improving alkali resistance or antibody acid dissociation properties, and for improving antibody-binding capacity by mutagenesis into the immobilization site (Patent Literatures 1 to 4).

Generally, protein variants obtained by introducing a plurality of mutations to proteins can be expressed at lower levels in cells than the wild-type proteins. It is known that the reduction in protein expression level can be prevented by adding a highly expressed protein (tag sequence) to the N-terminus to produce the target protein as a fusion protein. Such a fusion protein, however, contains the unnecessary tag sequence, which may reduce the activity of the target protein. The potential loss of activity of the target protein can be reduced by removing the tag sequence from the fusion protein. This, however, requires an additional step of removing the tag sequence and is therefore inefficient as a method for producing a protein (Non-Patent Literature 2, Patent Literature 5).

### CITATION LIST

### Patent Literature

Patent Literature 1: WO 2003/080655

Patent Literature 2: EP 1123389

Patent Literature 3: WO 2011/118699

Patent Literature 4: WO 2012/133349

Patent Literature 5: WO 2009/101672

### Non Patent Literature

Non-Patent Literature 1: Hober S. et al., “J. Chromatogr. B”, 2007, 848, pp. 40-47

Non-Patent Literature 2: Smith, D. B. et al., “Gene”, 1988, 67, pp. 31-40

One or more embodiments of the present invention relate to an immunoglobulin-binding engineered protein that can be produced by a transformant with improved culture productivity.

The inventors have designed a numerous recombinant variants of Protein A, obtained the variants by protein engineering and genetic engineering techniques, and evaluated the culture productivity of the variants. As a result, the inventors have found that a protein containing two or more linked domains derived from Protein A in which a mutation is introduced near the N-terminus of the domain closest to the N-terminus can be produced by a transformant with improved culture productivity, without deteriorating the properties of Protein A such as antibody-binding capacity and alkali resistance.

### SUMMARY

One or more embodiments of the present invention relate to a protein, having two or more amino acid sequences derived from any of the E, D, A, B, and C domains of Protein A of SEQ ID NOs:1 to 5, wherein in the domain-derived amino acid sequence closest to the N-terminus, one or more amino acid residues corresponding to position 4 and/or position 7 of the C domain are substituted by amino acid residues other than Arg, and wherein the protein can be produced by a transformant with improved culture productivity as compared to before the substitution.

In one or more embodiments, in the domain-derived amino acid sequence closest to the N-terminus, an amino acid residue corresponding to position 4 of the C domain is substituted by Gln, Ala, Gly, Leu, Met, Phe, Pro, Val, Trp, Asn, Cys, Ser, Thr, Tyr, Asp, Glu, His, or Lys.

In one or more embodiments, in the domain-derived amino acid sequence closest to the N-terminus, an amino acid residue corresponding to position 7 of the C domain is substituted by Met, Phe, Asn, Gln, Thr, Tyr, His, or Lys.

In one or more embodiments, in the domain-derived amino acid sequence second closest to the N-terminus or subsequent ones, Lys is substituted by an amino acid residue other than Lys.

In one or more embodiments, at least 90% of the following amino acid residues are retained: Gln-9, Gln-10, Phe-13, Tyr-14, Leu-17, Pro-20, Asn-21, Leu-22, Gln-26, Arg-27, Phe-30, Ile-31, Leu-34, Pro-38, Ser-39, Leu-45, Leu-51, Asn-52, Gln-55, and Pro-57, wherein the residue numbers indicated are for the C domain.

One or more embodiments of the present invention further relate to a DNA, encoding the protein.

One or more embodiments of the present invention further relate to a vector, including the DNA.

One or more embodiments of the present invention further relate to a transformant, produced by transforming a host cell with the vector.

One or more embodiments of the present invention further relate to a method for producing the protein, the method including using the transformant, or a cell-free protein synthesis system including the DNA.

One or more embodiments of the present invention further relate to an affinity separation matrix, including the protein as an affinity ligand immobilized on a carrier made of a water-insoluble base material.

In one or more embodiments, the affinity separation matrix binds to a protein containing an immunoglobulin Fc region.

In one or more embodiments of the affinity separation matrix, the protein containing an immunoglobulin Fc region is an immunoglobulin G or an immunoglobulin G derivative.

One or more embodiments of the present invention further relate to a method for preparing the affinity separation matrix, the method including immobilizing the protein as an affinity ligand onto a carrier made of a water-insoluble base material.

One or more embodiments of the present invention further relate to a method for purifying a protein containing an immunoglobulin Fc region, the method including adsorbing a polypeptide containing an immunoglobulin Fc region onto the affinity separation matrix.

The protein according to one or more embodiments of the present invention can be produced by a transformant with improved culture productivity and therefore can be produced economically and efficiently.

### BRIEF DESCRIPTION OF DRAWINGS

FIG. 1 shows a table for comparison of the sequences of the E, D, A, B, and C domains of Protein A from Staphylococcus sp.

### DETAILED DESCRIPTION OF EMBODIMENTS

The protein according to one or more embodiments of the present invention is a protein having two or more amino acid sequences derived from any of the E, D, A, B, and C domains of Protein A of SEQ ID NOs:1 to 5, wherein in the domain-derived amino acid sequence closest to the N-terminus, one or more amino acid residues corresponding to position 4 and/or position 7 of the C domain are substituted by amino acid residues other than Arg, and wherein the protein can be produced by the transformant with improved culture productivity as compared to before the substitution.

Substitutions of amino acid residues are denoted herein with the code for the wild-type or non-mutated type amino acid residue, followed by the position number of the substitution, followed by the code for changed amino acid residue. For example, a substitution of Ala for Gly at position 29 is represented by G29A.

The term “protein” is intended to include any molecule having a polypeptide structure and also encompass fragmented polypeptide chains and polypeptide chains linked by peptide bonds.

In general, the term “domain” refers to a higher-order protein structural unit having a sequence that consists of several tens to hundreds of amino acids, enough to fulfill a certain physicochemical or biochemical function. The domain as used herein particularly refers to a domain that binds to a protein containing an immunoglobulin Fc region.

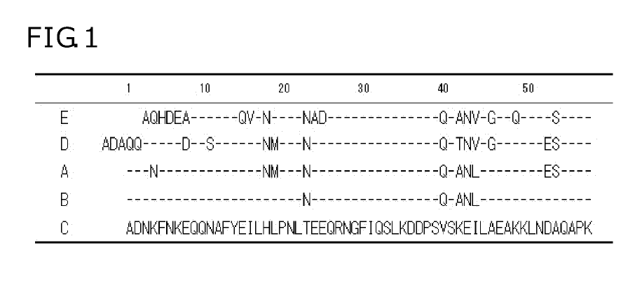

The domain-derived amino acid sequence refers to an amino acid sequence before the amino acid substitution. The domain-derived amino acid sequence is not limited only to the wild-type amino acid sequences of the E, D, A, B, and C domains of Protein A, and may include any amino acid sequence partially engineered by amino acid substitution, insertion, deletion, or chemical modification, provided that it forms a protein having the ability to bind to an Fc region. Examples of the domain-derived amino acid sequence include the amino acid sequences of the E, D, A, B, and C domains of Staphylococcus Protein A of SEQ ID NOs: 1 to 5. Examples also include proteins having amino acid sequences obtained by introducing a substitution of Ala for Gly corresponding to position 29 of the C domain into the E, D, A, B, and C domains of Protein A. In addition, the Z domain produced by introducing A1V and G29A mutations into the B domain corresponds to the domain-derived amino acid sequence because it also has the ability to bind to an Fc region. The domain-derived amino acid sequence may be a domain having high chemical stability or a variant thereof. These domains can be aligned as shown in FIG. 1. For example, the residue corresponding position 31 of the C domain corresponds to that at the same position 31 of the A or B domain, at position 29 of the E domain, and at position 34 of the D domain.

The amino acid sequence derived from any of the domains may be an amino acid sequence obtained by introducing into any of the amino acid sequences of SEQ ID Nos:1 to 5 an amino acid substitution that meets at least one of the following conditions (1) to (4):
(1) the amino acid residue in the corresponding domain that corresponds to position 29 of the C domain is Ala, Val, Leu, Ile, Phe, Tyr, Trp, Thr, Ser, Asp, Glu, Arg, His, or Met;
(2) the amino acid residue in the corresponding domain that corresponds to position 33 of the C domain is Leu, Ile, Phe, Tyr, Trp, Thr, Asp, Glu, Asn, Gln, Arg, His, or Met;
(3) the amino acid residue in the corresponding domain that corresponds to position 36 of the C domain is Leu, Ile, Phe, Tyr, Trp, Glu, Arg, His, or Met; and
(4) the amino acid residue in the corresponding domain that corresponds to position 37 of the C domain is Leu, Ile, Phe, Tyr, Trp, Glu, Arg, His, or Met.

The amino acid sequence derived from any of the domains may be an amino acid sequence obtained by introducing into any of the amino acid sequences of SEQ ID Nos:1 to 5 an amino acid substitution that meets at least one of the following conditions (1) to (4):
(1) the amino acid residue in the corresponding domain that corresponds to position 29 of the C domain is Ala, Glu, or Arg;
(2) the amino acid residue in the corresponding domain that corresponds to position 33 of the C domain is Leu, Thr, Glu, Gln, Arg, or His;
(3) the amino acid residue in the corresponding domain that corresponds to position 36 of the C domain is Ile or Arg; and
(4) the amino acid residue in the corresponding domain that corresponds to position 37 of the C domain is Leu, Ile, Glu, Arg, or His.

The domain-derived amino acid sequence may have at least 85%, at least 90%, or at least 95% sequence identity to any of the E, D, A, B, and C domains of Protein A of SEQ ID Nos:1 to 5.

The protein according to one or more embodiments of the present invention has two or more amino acid sequences derived from any of the E, D, A, B, and C domains of Protein A of SEQ ID Nos:1 to 5. The number of domains in the protein according to one or more embodiments of the present invention may be two or more, three or more, four or more, five or more, or six or more. The number of domains in the protein according to one or more embodiments of the present invention may be 20 or less, 10 or less, eight or less, or six or less.

In one or more embodiments of the present invention, the amino acid sequence of the domain closest to the N-terminus may be different from that of the domain(s) connected to the C-terminal end of the former domain. In the case where a plurality of domains are connected to the C-terminal end of the domain closest to the N-terminus, the domains other than the domain closest to the N-terminus may form a protein that includes a homopolymer (e.g. homodimer, homotrimer) consisting of linked single immunoglobulin-binding domains, terminated with Lys, or a protein that includes a heteropolymer (e.g. heterodimer, heterotrimer) consisting of linked different immunoglobulin-binding domains, terminated with Lys.

The monomeric proteins may be linked to each other by, for example, but not limited to: a method that does not use an amino acid residue as a linker; or a method that uses one or more amino acid residues. The number of amino acid residues used as the linker is not particularly limited, and may be such that the three-dimensional conformation of the monomeric proteins does not become unstable.

The term “linker” herein means a linker between domains, and refers to a linking portion of linked monomeric proteins (single domains), i.e., a region between the C-terminal region of a domain sequence closer to the N-terminus and the N-terminal region of a domain sequence closer to the C-terminus. In the case of a protein including N number of domains linked in tandem, the number of linkers in the protein is N-1. In other words, the “linker” herein refers to a region that consists of at least two amino acid residues, including the C-terminal amino acid of a domain closer to the N-terminus and the N-terminal amino acid of a domain closer to the C-terminus, which are connected to each other.

The linker may be an N-/C-terminal sequence of a domain that does not assume a specific secondary structure or that is located on the border between domains. In this case, the linker on the N-terminal side of an immunoglobulin G-binding domain of Protein A, for example, includes amino acid residues corresponding to positions 1 to 6, positions 1 to 5, positions 1 to 4, positions 1 to 3, or positions 1 to 2, of the C domain, and contains at least the N-terminal amino acid residue. It should be noted that although the E and D domains are different from the C domain in full length, their amino acid residues corresponding to the above-mentioned amino acids of the C domain correspond to the linker. Likewise, the linker on the C-terminal side of an immunoglobulin G-binding domain of Protein A, for example, includes amino acid residues corresponding to positions 55 to 58, positions 56 to 58, or positions 57 to 58, of the C domain, and contains at least the C-terminal amino acid residue at position 58.

The protein according to one or more embodiments of the present invention having two or more domains is such that in the domain-derived amino acid sequence closest to the N-terminus, one or more amino acid residues corresponding to position 4 and/or position 7 of the C domain are substituted by amino acid residues other than Arg. The amino acid substitution means a mutation which deletes the original amino acid and adds a different type of amino acid to the same position.

In the domain-derived amino acid sequence closest to the N-terminus, the amino acid residue corresponding to position 4 of the C domain to be substituted may be an amino acid residue at position 2 of the E domain of Protein A of SEQ ID No:1, an amino acid residue at position 7 of the D domain of Protein A of SEQ ID No:2, an amino acid residue at position 4 of the A domain of Protein A of SEQ ID No:3, an amino acid residue at position 4 of the B domain of Protein A of SEQ ID No:4, or an amino acid residue at position 4 of the C domain of Protein A of SEQ ID No:5.

In the domain-derived amino acid sequence closest to the N-terminus, the amino acid residue corresponding to position 7 of the C domain to be substituted may be an amino acid residue at position 5 of the E domain of Protein A of SEQ ID No:1, an amino acid residue at position 10 of the D domain of Protein A of SEQ ID No:2, an amino acid residue at position 7 of the A domain of Protein A of SEQ ID No:3, an amino acid residue at position 7 of the B domain of Protein A of SEQ ID No:4, or an amino acid residue at position 7 of the C domain of Protein A of SEQ ID No:5. The term “corresponding” means that they are in the same column when the E, D, A, B, and C domains of Protein A are aligned as shown in FIG. 1.

In the domain-derived amino acid sequence closest to the N-terminus, the amino acid residue other than Arg after substitution of the amino acid residue corresponding to position 4 of the C domain may be Gln, Ala, Gly, Leu, Met, Phe, Pro, Val, Trp, Asn, Cys, Ser, Thr, Tyr, Asp, Glu, His, or Lys (for example, Gln), Ala, Gly, Leu, Met, Phe, Pro, Val, Trp, Asn, Cys, Ser, Thr, Tyr, Asp, Glu, His, or Lys (for example, Gln or Lys).

In the domain-derived amino acid sequence closest to the N-terminus, the amino acid residue other than Arg after substitution of the amino acid residue corresponding to position 7 of the C domain may be Met, Phe, Asn, Gln, Thr, Tyr, His, or Lys (for example, Met), Phe, Asn, Gln, Thr, Tyr, His, or Lys (for example, Thr or Lys).

In the domain-derived amino acid sequence closest to the N-terminus, the amino acid substitutions at positions corresponding to positions 4 and 7 of the C domain may be introduced alone or in combination. In one or more embodiments, combinations of mutations include the following combinations (1) to (4):
(1) Position 4: Lys, position 7: Lys;
(2) Position 4: Gln, position 7: Lys;
(3) Position 4: Gln, position 7: Thr;
(4) Position 4: Lys, position 7: Thr.

In the domain-derived amino acid sequence closest to the N-terminus, the amino acid residues other than those corresponding to positions 4 and 7 of the C domain are not particularly limited as long as the protein can be produced by the transformant with improved culture productivity as compared to before substitution. Moreover, the domain-derived amino acid sequence second closest to the N-terminus and subsequent ones are not particularly limited either as long as the protein can be produced by the transformant with improved culture productivity as compared to before substitution.

The domain-derived amino acid sequence closest to the N-terminus, except for positions corresponding to positions 4 and 7 of the C domain, and the domain-derived amino acid sequence second closest to the N-terminus and subsequent ones may be functional variants that are free of Lys, except for linkers between domains and the C-terminus. Examples of such variants include C-K04R/K07R/G29A/S33R/K35R/K42R/K49Q/K50R.5d of SEQ ID No:8 in the Sequence Listing of WO 2012/133349. The Lys refers to six Lys residues in the E, D, A, or B domain of Protein A that correspond to positions 4, 7, 35, 49, 50, and 58 of the C domain. Only for the C domain of Protein A, it refers to seven Lys residues at positions 4, 7, 35, 42, 49, 50, and 58. However, it is not limited to the above numbers if the non-mutated amino acid sequences are sequences that have undergone deletion, such as sequences containing deletions at positions 1 to 4.

The amino acid residues in the domain-derived amino acid sequence closest to the N-terminus, except for positions corresponding to positions 4 and 7 of the C domain, and the domain-derived amino acid sequence second closest to the N-terminus and subsequent ones may be any amino acid residues such as wild-type amino acid residues, non-protein-forming amino acid residues, or non-natural amino acid residues. For production by genetic engineering, wild-type amino acid residues can be suitably used. However, amino acid residues having in a side chain a functional group that is highly reactive in a coupling reaction for immobilization, such as cysteine (Cys) having a thiol group (—SH) in a side chain, are unsuitable as amino acid residues used for substitution.

The domain-derived amino acid sequence closest to the N-terminus, except for positions corresponding to positions 4 and 7 of the C domain, and the domain-derived amino acid sequence second closest to the N-terminus and subsequent ones may contain amino acid substitutions that improve various functions. Examples of such amino acid substitutions include amino acid substitutions which substitute amino acids other than Ala for one or more Gly residues in an amino acid sequence derived from any of the E, D, A, B, and C domains of Protein A, as disclosed in WO 2010/110288. Examples also include amino acid substitutions which introduce at least one amino acid substitution into the amino acid residues at positions 31 to 37 of the A, B, or C domain, amino acid residues at positions 29 to 35 of the E domain, or amino acid residues at positions 34 to 40 of the D domain in an amino acid sequence derived from at least one domain selected from the E, D, A, B, and C domains of Protein A, as disclosed in WO 2011/118699. These amino acid substitutions can reduce affinity for Fab regions to improve antibody elution properties.

Shown below are examples of amino acid substitutions that may be contained in the domain-derived amino acid sequence closest to the N-terminus, except for positions corresponding to positions 4 and 7 of the C domain, and the domain-derived amino acid sequence second closest to the N-terminus and subsequent ones.
(1) A substitution of Arg, Gln, or Asn (for example, Arg), for Lys in the D, B, or C domain, except the domain closest to the N-terminus, that corresponds to position 4 of the C domain.
(2) A substitution of Asp, Arg, or Glu (for example, Arg), for Lys in the D, A, B, or C domain, except the domain closest to the N-terminus, that corresponds to position 7 of the C domain.
(3) A substitution of Arg, Ile, or His (for example, Arg or His), for Lys in the E, D, A, B, or C domain that corresponds to position 35 of the C domain.
(4) A substitution of Arg for Lys corresponding to position 42 in the C domain.
(5) A substitution of Arg or Gln for Lys in the D, A, B, or C domain that corresponds to position 49 of the C domain.
(6) A substitution of Arg or His (for example, Arg), for Lys in the E, D, A, B, or C domain that corresponds to position 50 of the C domain.
(7) A substitution of Arg or Gly (for example, Arg), for Lys in the E, D, A, B, or C domain that corresponds to position 58 of the C domain. However, if the amino acid residue in question is at (near) the C-terminus of the ligand, Lys may be left without introducing a substitution as described above.

In the domain-derived amino acid sequence second closest to the N-terminus or subsequent ones, Lys may be substituted by an amino acid residue other than Lys. The number of Lys residues substituted by amino acid residues other than Lys, among the Lys residues corresponding to positions 4, 7, 35, 42, 49, 50, and 58 of the C domain, may be one or more, two or more, three or more, four or more, five or more, six or more, or all Lys residues are substituted.

The domain-derived amino acid sequence second closest to the N-terminus or subsequent ones in which Lys is substituted by an amino acid residue other than Lys may, for example, be the following amino acid sequence: RFX₁X₂EQQNAFYEILHX₃PNLTEEQRNX₄FIQX₅LX₆X₇X₈PSVSREX₉LAEAX₁₀X₁₁LND AQAPX₁₂ wherein X₁=D, E, N, or Q; X₂=E or R; X₃=L, M, or I; X₄=A, E, F, R, Y, or W; X₅=D, E, H, I, L, Q, R, S, T, or V; X₆=H, I, or R; X₇=D, I, or R; X₈=D or E; X₉=I, L, or V; X₁₀=R or Q; X₁₁=H or R; X₁₂=R, G, or K, as disclosed in WO 2012/133342. Moreover, it may be an amino acid sequence derived from any of the E, D, A, B, and C domains of Protein A which contains amino acid substitutions for all lysine residues (Lys) and which is terminated with Lys, as disclosed in WO 2012/133349. Moreover, it may be an amino acid sequence including two or more amino acid sequences derived from any domain selected from the E, D, A, B, and C domains of Protein A which contain amino acid substitutions for all lysine residues (Lys), wherein the amino acid sequences are connected to each other through a linker, and at least one linker contains a lysine residue (Lys) or a cysteine residue (Cys), as disclosed in WO 2014/046278.

In the domain-derived amino acid sequence closest to the N-terminus, the domain-derived amino acid sequence second closest to the N-terminus or subsequent ones, at least half of the substitutions for Lys may be substitutions with arginine (Arg). This is because Arg is a basic amino acid having similar properties to Lys, and a substitution of Lys with Arg causes a relatively small effect on the properties of the whole protein.

In one or more embodiments of the protein of the present invention, at least 90%, or at least 95%, of the following 20 amino acid residues are retained: Gln-9, Gln-10, Phe-13, Tyr-14, Leu-17, Pro-20, Asn-21, Leu-22, Gln-26, Arg-27, Phe-30, Ile-31, Leu-34, Pro-38, Ser-39, Leu-45, Leu-51, Asn-52, Gln-55, and Pro-57 (the residue numbers indicated are for the C domain).

Moreover, the amino acid sequence of the whole protein may have at least 85%, at least 90%, or at least 95% sequence identity to the amino acid sequence before mutagenesis. The sequence identity of amino acid sequences can be analyzed by a person skilled in the art by direct comparison of the sequences; specifically, it may be analyzed using commercially available sequence analysis software, for example.

The protein according to one or more embodiments of the present invention may be a fusion protein produced by fusion with another protein having a different function. Examples of the fusion protein include, but are not limited to, those fused with albumin or GST (glutathione S-transferase). The protein according to one or more embodiments of the present invention may also be one fused with a nucleic acid (e.g. a DNA aptamer), a drug (e.g. an antibiotic), or a polymer (e.g. polyethylene glycol (PEG)).

One or more embodiments of the present invention also relate to a DNA having a base sequence encoding the protein obtained as above. The DNA may be any DNA having a base sequence that is translated into the amino acid sequence of the protein. Such a DNA can be obtained by common known techniques, such as polymerase chain reaction (hereinafter abbreviated as PCR). Alternatively, it can be synthesized by known chemical synthesis techniques or may be available from DNA libraries. A codon in the base sequence of the DNA may be replaced with a degenerate codon, and the base sequence is not necessarily the same as the original base sequence, provided that the coding base sequence is translated into the same amino acids.

Site-directed mutagenesis for modifying the base sequence of the DNA may be performed by, for example, recombinant DNA technology or PCR as follows.

Specifically, in the case of mutagenesis by recombinant DNA technology, for example, if there are suitable restriction enzyme recognition sequences on both sides of a mutagenesis target site in the gene encoding the protein according to one or more embodiments of the present invention, a cassette mutagenesis method can be used in which these restriction enzyme recognition sequences are cleaved with the restriction enzymes to remove a region containing the mutagenesis target site, and a DNA fragment in which only the target site is mutated by chemical synthesis or other methods is then inserted.

In the case of site-directed mutagenesis by PCR, for example, a double primer method can be used in which PCR is performed using a double-stranded plasmid encoding the protein as a template and two synthetic oligo primers containing complementary mutations in the + and − strands.

The DNA encoding the protein having two or more amino acid sequences derived from any of the E, D, A, B, and C domains of Protein A of SEQ ID Nos:1 to 5 can be obtained by ligating DNAs encoding a single domain. For example, the ligation of DNAs may be accomplished by introducing an appropriate restriction enzyme recognition sequence into a base sequence, fragmenting the sequence with the restriction enzyme, and ligating the double-stranded DNA fragments using a DNA ligase. A single restriction enzyme recognition sequence or a plurality of different restriction enzyme recognition sequences may be introduced.

The method for preparing the DNA encoding the protein having two or more amino acid sequences derived from any of the E, D, A, B, and C domains of Protein A of SEQ ID Nos:1 to 5 is not limited to the methods described above. For example, it may be prepared by applying any of the mutagenesis methods to a DNA encoding Protein A (e.g., see WO 2006/004067). Here, if the base sequences each encoding a monomeric protein in the DNA encoding a polymeric protein are the same, then homologous recombination of the DNA may be induced in transformed host cells. For this reason, the ligated DNAs encoding a monomeric protein may have 90% or lower, or 85% or lower base sequence identity.

The “vector” according to one or more embodiments of the present invention includes a base sequence encoding the above-described protein or a partial amino acid sequence thereof, and a promoter that is operably linked to the base sequence to function in a host cell. Typically, the vector can be constructed by linking or inserting the above-described gene encoding the protein into an appropriate vector. The vector used for insertion of the gene is not particularly limited, provided that it is capable of autonomous replication in a host cell. The vector may be a plasmid DNA or phage DNA. For example, when Escherichia coli is used as a host cell, examples of the vector include pQE vectors (QIAGEN), pET vectors (Merck), and pGEX vectors (GE Healthcare, Japan). Examples of plasmid vectors useful for the expression of Brevibacillus genes include the known Bacillus subtilis vector pUB110 and pHY500 (JP H02-31682 A), pNY700 (JP H04-278091 A), pNU211R2L5 (JP H07-170984 A), pHT210 (JP H06-133782 A), and the shuttle vector pNCMO2 between Escherichia coli and Brevibacillus (JP 2002-238569 A).

The protein according to one or more embodiments of the present invention can be obtained as a fusion protein with a known protein that serves to assist protein expression or facilitate purification. Examples of such proteins include, but are not limited to, maltose-binding protein (MBP) and glutathione S-transferase (GST). The fusion protein can be produced using a vector that contains the DNA according to one or more embodiments of the present invention and a DNA encoding a protein such as MBP or GST ligated to each other.

In one or more embodiments, the “transformant” can be produced by introducing the recombinant vector according to one or more embodiments of the present invention into a host cell. The recombinant DNA may be introduced into a host cell by, for example, but not limited to: a calcium ion method, an electroporation method, a spheroplast method, a lithium acetate method, an agrobacterium infection method, a particle gun method, or a polyethylene glycol method. Moreover, in one or more embodiments, the obtained gene function may be expressed in a host cell, for example, by incorporating the gene into a genome (chromosome).

The host cell is not particularly limited, and examples suitable for low-cost mass production are Escherichia coli, Bacillus subtilis, and bacteria (eubacteria) of genera including Brevibacillus, Staphylococcus, Streptococcus, Streptomyces, and Corynebacterium.

The protein according to one or more embodiments of the present invention may be produced by culturing the above-described transformant in a medium to produce and accumulate the protein in the cultured cells (including the periplasmic space thereof) or in the culture medium (extracellularly), and collecting the desired protein from the culture.

Alternatively, the protein according to one or more embodiments of the present invention may be produced by culturing the above-described transformant in a medium to produce and accumulate a fusion protein containing the protein in the cultured cells (including the periplasmic space thereof) or in the culture medium (extracellularly), collecting the fusion protein from the culture, cleaving the fusion protein with an appropriate protease, and collecting the desired protein.

The transformant according to one or more embodiments of the present invention can be cultured in a medium according to common methods for culturing host cells. The medium used to culture the transformant may be any medium that allows for high yield and high efficiency production of the protein. Specifically, carbon and nitrogen sources such as glucose, sucrose, glycerol, polypeptone, meat extracts, yeast extracts, and casamino acids may be used. In addition, the medium may optionally be supplemented with inorganic salts such as potassium salts, sodium salts, phosphates, magnesium salts, manganese salts, zinc salts, or iron salts. When an auxotrophic host cell is used, nutritional substances necessary for its growth may be added. Moreover, antibiotics such as penicillin, erythromycin, chloramphenicol, or neomycin may optionally be added.

Furthermore, a variety of known protease inhibitors, i.e. phenylmethane sulfonyl fluoride (PMSF), benzamidine, 4-(2-aminoethyl)-benzenesulfonyl fluoride (AEBSF), antipain, chymostatin, leupeptin, pepstatin A, phosphoramidon, aprotinin, and ethylenediaminetetraacetic acid (EDTA), and/or other commercially available protease inhibitors may be added at appropriate concentrations in order to reduce the degradation or molecular-size reduction of the target protein caused by host-derived proteases present inside or outside the cells.

Furthermore, in order to ensure accurate folding of the protein according to one or more embodiments of the present invention, molecular chaperones such as GroEL/ES, Hsp70/DnaK, Hsp90, or Hsp104/C1pB may be used (for example, they may be allowed to coexist with the protein by, for example, co-expression or incorporation into a fusion protein). Other methods for ensuring accurate folding of the protein according to one or more embodiments of the present invention may also be used such as, but not limited to, adding an additive for assisting accurate folding to the medium or culturing at low temperatures.

Examples of media that can be used to culture the transformant obtained using Escherichia coli as a host include LB medium (1% triptone, 0.5% yeast extract, 1% NaCl), 2xYT medium (1.6% triptone, 1.0% yeast extract, 0.5% NaCl), and D medium (1.6% Select soytone, 1.0% yeast extract, 0.5% NaCl).

Examples of media that can be used to culture the transformant obtained using Brevibacillus as a host include TM medium (1% peptone, 0.5% meat extract, 0.2% yeast extract, 1% glucose, pH 7.0), 2SL medium (4% peptone, 0.5% yeast extract, 2% glucose, pH 7.2), and A medium (3.0% polypeptone, 0.5% yeast extract, 3% glucose, 0.01% magnesium sulfate, 0.001% iron sulfate, 0.001% manganese chloride, 0.0001% zinc chloride).

The cell is aerobically cultured at a temperature of 15° C. to 42° C., such as 20° C. to 37° C., for several hours to several days under aeration and stirring conditions to accumulate the protein according to one or more embodiments of the present invention in the cultured cells (including the periplasmic space thereof) or in the culture medium (extracellularly), followed by recovery of the protein. In some cases, the cell may be cultured anaerobically without air.

In the case where the recombinant protein is secreted, the produced recombinant protein can be recovered after the culture by separating the cultured cells from the supernatant containing the secreted protein by a common separation method such as centrifugation or filtration.

Also, in the case where the protein is accumulated in the cultured cells (including the periplasmic space thereof), the protein produced and accumulated in the cells can be recovered, for example, by collecting the cells from the culture medium, e.g. via centrifugation or filtration, followed by disrupting the cells, e.g. via sonication or French press, and/or solubilizing the protein with, for example, a surfactant.

The protein according to one or more embodiments of the present invention can be purified by methods such as affinity chromatography, cation or anion exchange chromatography, and gel filtration chromatography, used alone or in an appropriate combination.

Whether the purified product is the target protein may be confirmed by common methods such as SDS polyacrylamide gel electrophoresis, N-terminal amino acid sequencing, or Western blot analysis.

The protein according to one or more embodiments of the present invention can also be produced using a cell-free protein synthesis system including the DNA. Examples of such cell-free protein synthesis systems include synthesis systems derived from procaryotic cells, plant cells, or higher animal cells.

The protein according to one or more embodiments of the present invention is characterized in that it can be produced by the transformant with improved culture productivity as compared to before the amino acid substitution. The term “culture productivity” can be rephrased as “protein expression level” and refers to the amount per unit volume or per cell of the target recombinant protein produced by culturing the transformant as described above. For example, for secretory production, it may be the concentration of the target protein in the culture supernatant, while in the case where the target recombinant protein is produced in the cells, it can be expressed as the weight of the target protein per cell count or per cell weight. The expression “the protein can be produced by the transformant with improved culture productivity as compared to before the amino acid substitution” means that when a transformant producing a non-mutated protein and a transformant producing the protein containing amino acid substitution are cultured under the same conditions, and then evaluated by the later-described “Method for evaluating culture productivity”, the relative value is higher by at least 5%, for example, by at least 10% or by at least 20%.

The culture productivity can be evaluated as follows, for example.

### [Method for Evaluating Culture Productivity]

A transformant producing a non-mutated protein (control) and a transformant producing the protein containing amino acid substitution (evaluation sample) are prepared using the same host and the same transformation method, are cultured under the same conditions. For example, they are aerobically cultured in a medium suited for the host selected from the media mentioned above at an appropriate culture temperature (e.g. 20° C. to 37° C.) under aeration and stirring conditions for several hours to several days to accumulate the protein according to one or more embodiments of the present invention in the cultured cells (including the periplasmic space thereof) or in the culture medium (extracellular secretion), followed by recovery of the protein. The amount of the protein in the cultured cells or culture medium is quantitated. The quantitation may be carried out by high performance liquid chromatography (HPLC), for example. In HPLC analysis, an appropriate pretreatment is performed on the cultured cells or culture medium. For example, in the case where the protein is accumulated in the cells, the cells are disrupted with a device such as a sonicator and then centrifuged to remove the cell residues, followed by filtration. In the case where the protein is extracellularly secreted, the cells are separated from the culture medium by centrifugation, followed by filtration. The control, the evaluation sample, and a reference (a highly purified protein having a predetermined concentration) are analyzed by HPLC, and the concentration of the reference and the analytical data (chromatographic areas) are used to calculate the concentration of the protein in the control or evaluation sample, which is then used to calculate the relative value using the following (Equation 1). The culture productivity is considered improved if the relative value of the evaluation sample is higher by at least 5%, for example, by at least 10% or by at least 20% than that of the control.

Relative value (%)=[protein concentration of control]/[protein concentration of evaluation sample] (Equation 1)

The protein according to one or more embodiments of the present invention can be used as an affinity ligand having affinity for an immunoglobulin. An affinity separation matrix can be prepared by a method including immobilizing the protein according to one or more embodiments of the present invention as an affinity ligand onto a carrier made of a water-insoluble base material.

The term “affinity ligand” means a substance (functional group) that selectively captures (binds to) a target molecule from a mixture of molecules by virtue of a specific affinity between the molecules such as antigen-antibody binding, and refers herein to a protein that specifically binds to an immunoglobulin. The term “ligand” as used alone herein is synonymous with “affinity ligand”.

The “immunoglobulin binding properties” can be tested using, for example, but not limited to, biosensors such as Biacore system (GE Healthcare, Japan) based on the surface plasmon resonance principle. The measurement may be carried out under any conditions that allow detection of a binding signal corresponding to the binding of Protein A to an immunoglobulin Fc region. The properties can be easily evaluated at a (constant) temperature of 20° C. to 40° C. and a neutral pH of 6 to 8.

Examples of binding parameters that can be used include affinity constant (KA) and dissociation constant (KD) (Nagata et al., “Real-time analysis of biomolecular interactions”, Springer-Verlag Tokyo, 1998, page 41). The affinity constant of the protein according to one or more embodiments of the present invention for Fc may be determined in an experimental system using Biacore system in which human IgG is immobilized on a sensor chip, and each domain variant is added to a flow channel at a temperature of 25° C. and a pH of 7.4. The protein according to one or more embodiments of the present invention may suitably be a protein having an affinity constant (KA) for human IgG of at least 1×10⁵ (M⁻¹), at least 1×10⁶ (M⁻¹), or 1×10⁷ (M⁻¹) .

In order to provide an affinity ligand that has high chemical stability under alkali conditions, the amino acid sequence before mutagenesis may be an amino acid sequence derived from the C domain of SEQ ID No:5. Moreover, the amino acid sequence before mutagenesis may be an amino acid sequence derived from the C domain of SEQ ID No:5 in which the amino acid residue corresponding to position 29 is any amino acid residue selected from Ala, Arg, Glu, Ile, Leu, Met, Phe, Trp, and Tyr. Furthermore, at least half of the amino acid residues introduced to all Lys residues may be substitutions with Arg, or all of them may be substitutions with Arg. In the case where all of them are substitutions with Arg, the chemical stability under alkali conditions can be improved as compared to before the mutagenesis.

The chemical stability under alkali conditions may be determined using binding activity to an immunoglobulin or material stability of a polypeptide itself as an index. In the case where material stability of a polypeptide itself is used as an index, the chemical stability under alkali conditions can be evaluated, for example, by electrophoresis in which electrophoresis bands of the polypeptide before and after an alkali treatment are compared. Specifically, comparison of chemical stability may be made by performing standard SDS-PAGE to analyze band intensity via densitometry. If chemical stability is indicated based on the band intensities analyzed via densitometry, the polypeptide according to one or more embodiments of the present invention, after being left in a 0.5 M sodium hydroxide aqueous solution at 25° C. for 24 hours, may have a band intensity of 50% or higher, 60% or higher, 70% or higher, or 80% or higher, of that before the treatment.

Examples of the carrier made of a water-insoluble base material used in one or more embodiments of the present invention include inorganic carriers such as glass beads and silica gel; organic carriers such as synthetic polymers (e.g. cross-linked polyvinyl alcohol, cross-linked polyacrylate, cross-linked polyacrylamide, cross-linked polystyrene) and polysaccharides (e.g. crystalline cellulose, cross-linked cellulose, cross-linked agarose, cross-linked dextran); and composite carriers formed by combining these carriers, such as organic-organic or organic-inorganic composite carriers. Examples of commercially available products include GCL2000 (porous cellulose gel), Sephacryl S-1000 (prepared by covalently cross-linking allyl dextran with methylene bisacrylamide), Toyopearl (methacrylate carrier), Sepharose CL4B (cross-linked agarose carrier), and Cellufine (cross-linked cellulose carrier), although the water-insoluble carrier used in one or more embodiments of the present invention is not limited to the carriers listed above.

In view of the purpose and method of using the affinity separation matrix, the water-insoluble carrier used in one or more embodiments of the present invention should have a large surface area and may be a porous material having a large number of fine pores of an appropriate size. The carrier may be in any form such as bead, monolith, fiber, film (including hollow fiber) or other optional forms.

The ligand may be immobilized by any method that allows the ligand to be covalently bonded to the carrier via the s-amino group of lysine on the ligand by a conventional coupling process. Moreover, even if some ligands turn out to be immobilized on the carrier via the a-amino group at the N-terminus, the effect according to one or more embodiments of the present invention will not deteriorate because they are immobilized at the protein terminus. The coupling process may be carried out by reacting the carrier with cyanogen bromide, epichlorohydrin, diglycidyl ether, tosyl chloride, tresyl chloride, hydrazine, sodium periodate, or the like to activate the carrier (or introduce a reactive functional group into the carrier surface), and performing a coupling reaction between the carrier and the compound to be immobilized as a ligand to immobilize the ligand; or by adding a condensation reagent such as carbodiimide or a reagent having a plurality of functional groups in the molecule such as glutaraldehyde to a system containing the carrier and the compound to be immobilized as a ligand, followed by condensation and cross-linking for immobilization. Moreover, a spacer molecule consisting of a plurality of atoms may be introduced between the ligand and the carrier, or alternatively, the ligand may be directly immobilized onto the carrier.

The affinity separation matrix according to one or more embodiments of the present invention can be used to separate and purify a protein containing an immunoglobulin Fc region by affinity column chromatography purification techniques. As described earlier, the regions to which immunoglobulin-binding domains bind are broadly specified as Fab regions (particularly Fv regions) and Fc regions. However, since the conformation of antibodies is already known, it is possible to further alter (e.g. fragmentize) the Fab or Fc regions while maintaining the conformation of the regions to which Protein A binds by protein engineering techniques. Accordingly, the present invention is not limited to immunoglobulin molecules containing Fab and Fc regions sufficiently, and derivatives thereof. Therefore, the term “protein containing an immunoglobulin Fc region” refers to a protein containing an Fc region portion to which Protein A binds, and it does not have to contain the complete Fc region as long as Protein A is able to bind to the protein.

Typical examples of the protein containing an immunoglobulin Fc region include, but are not limited to, immunoglobulin G and immunoglobulin G derivatives. The term “immunoglobulin G derivative” is a generic term for engineered artificial proteins to which Protein A can bind, such as chimeric immunoglobulin G in which the domains of human immunoglobulin G are partially replaced and fused with immunoglobulin G domains of another biological species, humanized immunoglobulin G in which complementarity determining regions (CDRs) of human immunoglobulin G are replaced and fused with antibody CDRs of another biological species, immunoglobulin G whose Fc region has a molecularly altered sugar chain, and artificial immunoglobulin G in which the Fv and Fc regions of human immunoglobulin G are fused.

These proteins containing an immunoglobulin Fc region can be purified according to affinity column chromatographic purification techniques using existing commercial Protein A columns. Specifically, a buffer containing the protein containing an immunoglobulin Fc region is adjusted to be neutral, and the resulting solution is passed through an affinity column filled with the affinity separation matrix according to one or more embodiments of the present invention to adsorb the protein containing an immunoglobulin Fc region. Next, an appropriate volume of pure buffer is passed through the affinity column to wash the inside of the column. At this point, the desired protein containing an immunoglobulin Fc region remains adsorbed on the affinity separation matrix according to one or more embodiments of the present invention in the column. Subsequently, an acidic buffer (which may contain a substance for promoting dissociation from the matrix) adjusted to an appropriate pH is passed through the column to elute the desired protein containing an immunoglobulin Fc region, whereby high-level purification can be achieved.

The affinity separation matrix according to one or more embodiments of the present invention can be reused by passing therethrough a pure buffer having an appropriate strong acidity or strong alkalinity which does not completely impair the functions of the ligand compound and the carrier base material (or optionally a solution containing an appropriate modifying agent or an organic solvent) for washing.

One or more embodiments of the present invention further relate to a protein obtained by a separation method using the affinity separation matrix. The protein may be a protein containing an immunoglobulin Fc region. The protein obtained by using the affinity separation matrix is obtained as a high-purity, high-concentration solution, and has properties that maintain its inherent activity such as ability to bind to an antigen, without impairing it.

### EXAMPLES

The following description is offered to illustrate one or more embodiments of the present invention in greater detail by reference to examples, but the scope of the present invention is not limited to these examples. In the examples, operations such as recombinant DNA production and engineering were performed in accordance with the following textbooks, unless otherwise noted: (1) T. Maniatis, E. F. Fritsch, J. Sambrook, “Molecular Cloning/A Laboratory Manual”, 2nd edition (1989), Cold Spring Harbor Laboratory (USA); (2) Masami Muramatsu, “Lab Manual for Genetic Engineering”, 3rd edition (1996), Maruzen Co., Ltd. The materials such as reagents and restriction enzymes used in the examples were commercially available products, unless otherwise specified.

Proteins obtained in the examples are represented by “an alphabetical letter identifying the domain—an introduced mutation (wild for the wild type)”. For example, the wild-type C domain of Protein A is represented by “C-wild”, and a C domain variant containing G29E mutation is represented by “C-G29E”. Variants containing two mutations together are represented by indicating both with a slash. For example, a C domain variant containing G29E and S13L mutations is represented by “C-G29E/S13L”. Proteins consisting of a plurality of single domains linked together are represented by adding a period (.) followed by the number of linked domains followed by “d”. For example, a protein consisting of five linked C domain variants containing G29E and S13L mutations is represented by “C-G29E/S13L.5d”. In the case of proteins containing a mutation in the domain closest to the N-terminus, and a different mutation in the domain second closest to the N-terminus and subsequent domains, the mutations in the second and subsequent domains are represented as described above, and the mutation in the domain closest the N-terminus is added after the number of linked domains. For example, a protein consisting of five linked C domain variants and containing K04Q mutation in the domain closest to the N-terminus and KO4R mutation in the domains second to fifth closest to the N-terminus is represented by “C-K04R.5d-K04Q”.

### Example 1

### Evaluation of C Domain Variant Containing Amino Acid Substitution at Position 4

Expression plasmids of C domain variants of Protein A obtained by substituting Lys residues other than the C-terminal Lys-58 by other amino acids (C-K04R/K07R/G29A/S33R/K35R/K42R/K49Q/K50R.2d (SEQ ID No:6) and C-K04Q/K07R/G29A/S33R/K35R/K42R/K49Q/K5OR.2d (SEQ ID No:7)) were prepared by the following procedure.

The total synthesis of a DNA (SEQ ID No:8) encoding C-K04R/K07R/G29A/S33R/K35R/K42R/K49Q/K5OR.2d in which PstI and XbaI recognition sites were added to the 5′ and 3′ ends, respectively, was outsourced to Eurofins Genomics K.K. It was subcloned into an expression plasmid, which was then digested with the restriction enzymes PstI and XbaI (Takara Bio, Inc.), and the obtained DNA was ligated to a Brevibacillus expression vector pNCMO2 (Takara Bio, Inc.) digested with the same restriction enzymes to construct an expression plasmid in which a DNA encoding the amino acid sequence of SEQ ID No:6 was inserted into the Brevibacillus expression vector pNCMO2. The plasmid was prepared using Escherichia coli JM109.

The total synthesis of a DNA (SEQ ID No:9) encoding C-K04Q/K07R/G29A/S33R/K35R/K42R/K49Q/K5OR.2d (SEQ ID No:7) was similarly performed to prepare a plasmid.

Brevibacillus choshinensis SP3 (Takara Bio, Inc.) was transformed with the obtained plasmids, and the recombinant strains secreting the C domain variants were grown. Each recombinant strain was cultured with shaking at 30° C. for three days in 30 mL of A medium (3.0% polypeptone, 0.5% yeast extract, 3% glucose, 0.01% magnesium sulfate, 0.001% iron sulfate, 0.001% manganese chloride, 0.0001% zinc chloride) containing 60 pg/mL neomycin. After the culture, the culture medium was sampled to analyze turbidity at 600 nm using a spectrophotometer. Moreover, the cells were removed from the culture medium by centrifugation (15,000 rpm, 25° C., five minutes) to measure the concentration of the C domain variant in the culture supernatant by high performance liquid chromatography. The culture results are shown in Table 1.

The variant C-K04Q/K07R/G29A/S33R/K35R/K42R/K49Q/K50R.2d was produced with higher culture productivity (higher concentration of the C domain variant in the culture supernatant) than C-K04R/K07R/G29A/S33R/K35R/K42R/K49Q/K50R.2d. The amino acid substitution at position 4 of the C domain resulted in improved culture productivity of the transformant.

TABLE 1

| | | Concentration of |
|---|---|---|
| | | C domain variant |
| C domain variant | Turbidity | (g/L) |
| C-K04R/K07R/G29A/S33R/K35R/K42R/ | 32 | 1.1 |
| K49Q/K50R.2d | | |
| C-K04Q/K07R/G29A/S33R/K35R/K42R/ | 33 | 1.7 |
| K49Q/K50R.2d | | |

### Example 2

### Evaluation of C Domain Variant Containing Amino Acid Substitution at Position 4 of N-Terminal Domain

The total synthesis of a DNA (SEQ ID No:11) encoding C-K04R/K07R/G29A/S33R/K35R/K42R/K49Q/K50R.5d (SEQ ID No:10) in which PstI and XbaI recognition sites were added to the 5′ and 3′ ends, respectively, was outsourced to Eurogentec. The DNA was used to prepare a plasmid and grow a recombinant strain as in Example 1.

PCR was performed using the DNA encoding C-K04R/K07R/G29A/S33R/K35R/K42R/K49Q/K50R.5d as a template and Primer 1: 5′-TTCGCTGCAGATAACCAATTTAACCGTGAA-3′ (SEQ ID No:12) and Primer 2: 5′-ACTATCTAGATTATTTTGGAGCTTGTGCAT-3′ (SEQ ID No:13) to amplify a DNA fragment encoding C-K04R/K07R/G29A/S33R/K35R/K42R/K49Q/K50R.5d-K04Q in which K04Q substitution was introduced only into the N-terminal domain of C-K04R/K07R/G29A/S33R/K35R/K42R/K49Q/K50R.5d. The DNA fragment was digested with restriction enzymes PstI and XbaI, and ligated to a Brevibacillus expression vector pNCMO2 digested with the same restriction enzymes to construct an expression plasmid in which the DNA encoding C-K04R/K07R/G29A/S33R/K35R/K42R/K49Q/K50R.5d-K04Q was inserted into the Brevibacillus expression vector pNCMO2. The plasmid was sequenced using BigDye Terminator Cycle Sequencing Kit (Life Technologies Japan Ltd.) and Applied Biosystems 3130x1 genetic analyzer (Life Technologies Japan Ltd.) to confirm that the mutation was introduced only at the target position. Similarly to Example 1, Brevibacillus choshinensis SP3 was transformed and the recombinant strain secreting C-K04R/K07R/G29A/S33R/K35R/K42R/K49Q/K50R.5d-K04Q was grown.

The transformant producing C-K04R/K07R/G29A/S33R/K35R/K42R/K49Q/K50R.5d and the transformant producing C-K04R/K07R/G29A/S33R/K35R/K42R/K49Q/K50R.5d-K04Q were cultured as in Example 1 and the culture media were evaluated (n=3). The results (averages with n=3) are shown in Table 2.

The variant C-K04R/K07R/G29A/S33R/K35R/K42R/K49Q/K50R.5d-K04Q was produced with higher culture productivity than C-K04R/K07R/G29A/S33R/K35R/K42R/K49Q/K50R.5d. The amino acid substitution only at position 4 of the N-terminal domain, among the five domains, resulted in improved culture productivity.

TABLE 2

| | | Concentration of |
|---|---|---|
| | | C domain variant |
| C domain variant | Turbidity | (g/L) |
| C-K04R/K07R/G29A/S33R/K35R/K42R/ | 42 | 2.0 |
| K49Q/K50R.5d | | |
| C-K04R/K07R/G29A/S33R/K35R/K42R/ | 36 | 2.6 |
| K49Q/K50R.5d-K04Q | | |

### Example 3

### Evaluation of Culture of C Domain Variant Containing Amino Acid Substitutions at Position 4/Position 7 of N-Terminal Domain (Codon Engineering)

Similarly to Example 1, four mutants were produced which had different arginine codons at position 4 or 7 of the N-terminal domain in C-K04R/K07R/G29A/S33R/K35R/K42R/K49Q/K50R.5d. Also, mutants were produced which had different glutamine codons at position 4 of the N-terminal side domain in C-K04R/K07R/G29A/S33R/K35R/K42R/K49Q/K50R.5d-K04Q. For PCR primers, the total synthesis of five DNAs (Table 3) obtained by substituting a base sequence encoding the amino acid at position 4 or 7 in the sequence of Primer 1 shown in Example 1 was outsourced to Eurofins Genomics K.K. Transformants producing the variants were cultured as in Example 1 and the culture media were evaluated (n=3). The results (averages with n=3) are shown in Table 3.

There was not much difference in culture productivity of C-K04R/K07R/G29A/S33R/K35R/K42R/K49Q/K50R.5d before and after the codon engineering, and the culture productivity of C-K04R/K07R/G29A/S33R/K35R/K42R/K49Q/K50R.5d-K04Q was still high even when the codon was engineered. These results show that culture productivity is not affected by codon engineering, but by amino acid substitution.

TABLE 3

| Mutation in | | | Concentration of |
|---|---|---|---|
| N-terminal domain | | | C domain variant |
| Position 4 (codon) | Position 7 (codon) | Turbidity | (g/L) |
| R (CGT) | R (CGT) | 40 | 1.9 |
| R (CGC) | R (CGT) | 42 | 1.9 |
| R (AGA) | R (CGT) | 43 | 2.0 |
| R (CGT) | R (CGC) | 41 | 2.0 |
| R (CGT) | R (AGA) | 41 | 1.9 |
| Q (CAA) | R (CGT) | 39 | 2.6 |
| Q (CAC) | R (CGT) | 38 | 2.5 |

### Example 4

### Evaluation of Culture of C Domain Variant Containing Amino Acid Substitution at Position 4/Position 7 of N-Terminal Domain

Similarly to Example 1, 37 variants were produced which contained amino acid substitutions at position 4 or 7 of the N-terminal domain in C-K04R/K07R/G29A/S33R/K35R/K42R/K49Q/K50R.5d (Table 4). For PCR primers, the total synthesis of 37 DNAs obtained by substituting a base sequence encoding the amino acid at position 4 or 7 in the sequence of Primer 1 shown in Example 1 was outsourced to Eurofins Genomics K.K. The variants were cultured and evaluated as in Example 1.

The culture productivity of the variants was evaluated based on the values relative to the concentration of the C domain variant in the culture supernatant of C-K04R/K07R/G29A/S33R/K35R/K42R/K49Q/K50R.5d taken as 100%. The results are shown in Table 4.

The culture productivity was improved for the multiple variants, as well as C-K04R/K07R/G29A/S33R/K35R/K42R/K49Q/K50R.5d-K04Q shown in Example 1.

TABLE 4

| | Mutation in | | Culture |
|---|---|---|---|
| | N-terminal domain | | productivity |
| No. | Position 4 | Position 7 | relative value (%) |
| Before mutagenesis | R | R | 100 |
| 1 | A | R | 111 |
| 2 | G | R | 114 |
| 3 | I | R | 102 |
| 4 | L | R | 118 |
| 5 | M | R | 133 |
| 6 | F | R | 112 |
| 7 | P | R | 116 |
| 8 | V | R | 156 |
| 9 | W | R | 186 |
| 10 | N | R | 180 |
| 11 | C | R | 219 |
| 12 | S | R | 117 |
| 13 | T | R | 149 |
| 14 | Y | R | 132 |
| 15 | D | R | 196 |
| 16 | E | R | 258 |
| 17 | H | R | 110 |
| 18 | K | R | 143 |
| 19 | R | A | 86 |
| 20 | R | G | 81 |
| 21 | R | I | 86 |
| 22 | R | L | 75 |
| 23 | R | M | 224 |
| 24 | R | F | 128 |
| 25 | R | P | 86 |
| 26 | R | V | 60 |
| 27 | R | W | 98 |
| 28 | R | N | 169 |
| 29 | R | C | 82 |
| 30 | R | Q | 137 |
| 31 | R | S | 98 |
| 32 | R | T | 217 |
| 33 | R | Y | 106 |
| 34 | R | D | 84 |
| 35 | R | E | 100 |
| 36 | R | H | 136 |
| 37 | R | K | 205 |

### Example 5

### Evaluation of Mini Jar Culturing of C Domain Variant Containing Amino Acid Substitution at Position 4/Position 7 of N-Terminal Domain

Transformants producing the N-terminal variants C-K04R/K07R/G29A/S33R/K35R/K42R/K49Q/K50R.5d-K04Q (hereinafter, referred to as KO4Q) and C-K04R/K07R/G29A/S33R/K35R/K42R/K49Q/K50R.5d-K07T (hereinafter, referred to as K07T), which were prepared in Examples 2 and 3 and exhibited improved culture productivity, and C-K04R/K07R/G29A/S33R/K35R/K42R/K49Q/K50R.5d (hereinafter, referred to as control) were aerobically cultured using a 5 L mini jar in C medium (1.5% peptone, 0.8% yeast extract, 2.0% glucose, 0.38% phosphate, 0.04% magnesium sulfate heptahydrate, 0.004% manganese sulfate pentahydrate, 0.004% iron sulfate heptahydrate, 0.0004% zinc sulfate heptahydrate, 750 ppm Disfoam CC-118, pH 7.2; glucose continuously added (up to 3.8%) from 6 h after the start of the culture). The culture temperature was 34° C. from the start of the culture to 13.5 h and then shifted to 30° C. The pH of the medium was controlled in the range of pH 7.0 to pH 7.2 from the start to the end of the culture. The pH for the control was controlled in the range of pH 7.9 to pH 8.0 to provide higher culture productivity. The culture media were sampled over time to measure the turbidity and the concentration of the C domain variant in the culture supernatant as in Example 1. The results after 48 hours of culture are shown in Table 5.

The N-terminal variants were also produced with higher culture productivity in mini jar culture than the non-mutated protein.

TABLE 5

| | | Concentration of C |
|---|---|---|
| C domain variant | Turbidity | domain variant (g/L) |
| Control | 81 | 4.0 |
| K04Q | 57 | 5.5 |
| K07T | 58 | 5.9 |

### Example 6

### Evaluation of Culture of C Domain Variant Containing Multiple Amino Acid Substitutions at Position 4/Position 7 of N-Terminal Domain

Similarly to Example 3, four variants were prepared which contained multiple amino acid substitutions at positions 4 and 7 of the N-terminal domain in C-K04R/K07R/G29A/S33R/K35R/K42R/K49Q/K50R.5d (Table 6). For PCR primers, the total synthesis of four DNAs obtained by substituting base sequences encoding the amino acids at positions 4 and 7 in the sequence of Primer 1 shown in Example 1 was outsourced to Eurofins Genomics K.K. The variants were cultured and evaluated as in Example 1.

The culture productivity for each variant was evaluated based on the C domain variant concentration in the culture supernatant of C-K04R/K07R/G29A/S33R/K35R/K42R/K49Q/K50R.5d (n=3). The results (averages with n=3) are shown in Table 6.

Each multiple substitution variant was produced with improved culture productivity as compared to C-K04R/K07R/G29A/S33R/K35R/K42R/K49Q/K50R.5d.

TABLE 6

| | | Concentration of C |
|---|---|---|
| C domain variant | Turbidity | domain variant (g/L) |
| C-K04R/K07R/G29A/S33R/K35R/ | 42 | 2.0 |
| K42R/K49Q/K50R.5d | | |
| C-K04R/K07R/G29A/S33R/K35R/ | 38 | 2.3 |
| K42R/K49Q/K50R.5d-4K7K | | |
| C-K04R/K07R/G29A/S33R/K35R/ | 37 | 2.5 |
| K42R/K49Q/K50R.5d-4Q7K | | |
| C-K04R/K07R/G29A/S33R/K35R/ | 34 | 2.9 |
| K42R/K49Q/K50R.5d-4Q7T | | |
| C-K04R/K07R/G29A/S33R/K35R/ | 35 | 2.9 |
| K42R/K49Q/K50R.5d-4K7T | | |

### Example 7

### Evaluation of Culture of C Domain Variant Containing Amino Acid Substitution at Position 7 of N-Terminal Domain

Plasmids were constructed using a DNA (SEQ ID No:15) encoding the C domain variant C-G29A.2d (SEQ ID No:14) and a DNA (SEQ ID No:17) encoding the N-terminal domain variant C-G29A.2d-K07T (SEQ ID No:16) and transformants were grown as in Example 1. The total synthesis of each DNA in which Pstl and Xbal recognition sites were added to the 5′ and 3′ ends, respectively, was outsourced to Eurofins Genomics K.K. Each transformant was cultured and evaluated as in Example 1 (n=3). The results (averages with n=3) are shown in Table 7.

The variant C-G29A.2d-K07T was produced with higher culture productivity than C-G29A.2d. This shows that the culture productivity-improving effect of N-terminal mutation is not limited to C domain variants of C-K04R/K07R/G29A/S33R/K35R/K42R/K49Q/K50R.

TABLE 7

| | | Concentration of C |
|---|---|---|
| C domain variant | Turbidity | domain variant (g/L) |
| C-G29A.2d | 37 | 2.5 |
| C-G29A.2d-K07T | 35 | 2.7 |

### Example 8

### Evaluation of Culture in Escherichia coli of C Domain Variant Containing Amino Acid Substitution at Position 4/Position 7 of N-Terminal Domain

The total synthesis of a DNA (SEQ ID No:11) encoding C-K04R/K07R/G29A/S33R/K35R/K42R/K49Q/K50R.5d (SEQ ID No:10) in which PstI and XbaI recognition sites were added to the 5′ and 3′ ends, respectively, was outsourced to Eurogentec. PCR was performed using the DNA as a template, Primer 3: 5′-TTCGCATATGGCAGATAACCGTTTTAACCGTGAAC-3′ (SEQ ID No:18), and Primer 4: 5′-TTTTCTGCAGTTATTATTTTGGAGCTTGTGCATCA-3′ (SEQ ID No:19) to amplify the DNA in which Met and an NdeI recognition site were added to the 5′ end and a PstI recognition site to the 3′ end. Further, Primer 3 was replaced by Primer 5: 5′-TTCGCATATGGCAGATAACCAATTTAACCGTGAAC-3′ (SEQ ID No:20), Primer 6: 5′-TTCGCATATGGCAGATAACCGTTTTAACACTGAAC-3′ (SEQ ID No:21), and Primer 7: 5′-TTCGCATATGGCAGATAACCAATTTAACACTGAAC-3′ (SEQ ID No:22) to amplify, by PCR, C-K04R/K07R/G29A/S33R/K35R/K42R/K49Q/K50R.5d-K04Q, C-K04R/K07R/G29A/S33R/K35R/K42R/K49Q/K50R.5d-K07T, and C-K04R/K07R/G29A/S33R/K35R/K42R/K49Q/K50R.5d-K04Q/K07T, respectively.

The obtained DNA fragments were digested with restriction enzymes Ndel and Pstl, and ligated to an Escherichia coli expression vector pUCNT (WO 94/03613) digested with the same restriction enzymes to construct expression plasmids in which a DNA encoding each C domain variant was inserted into the Escherichia coli expression vector pUCNT. The base sequences in the plasmids were determined using BigDye Terminator Cycle Sequencing Kit (Life Technologies Japan Ltd.) and Applied Biosystems 3130×1 genetic analyzer (Life Technologies Japan Ltd.) to confirm that the mutation was introduced only at the target position. Escherichia coli HB101 (Takara Bio, Inc.) was transformed with the obtained plasmids, and the transformants producing each C domain variant were grown.

The transformants producing each C domain variant were cultured with shaking at 37° C. for 24 hours in 50 mL of D medium (1.6% Select soytone, 1.0% yeast extract, 0.5% NaCl) containing 100 μg/mL ampicillin. After the culture, the culture media were sampled to analyze turbidity at 600 nm using a spectrophotometer. Each culture medium was centrifuged to remove the supernatant and obtain the cells, which were then disrupted by an ultrasonic homogenizer, and the homogenate was centrifuged to obtain a cell-free extract. The concentration of the C domain variant in the cell-free extract was measured by high performance liquid chromatography. The culture was performed with n=3. The results (averages with n=3) are shown in Table 8.

Each N-terminal variant was also produced by a recombinant Escherichia coli with higher culture productivity than C-K04R/K07R/G29A/S33R/K35R/K42R/K49Q/K50R.5d. This shows that the culture productivity-improving effect of N-terminal mutation is not limited to the cases using Brevibacillus host cells.

TABLE 8

| | | Concentration of C |
|---|---|---|
| C domain variant | Turbidity | domain variant (g/L) |
| C-K04R/K07R/G29A/S33R/K35R/ | 18 | 0.8 |
| K42R/K49Q/K50R.5d (control) | | |
| C-K04R/K07R/G29A/S33R/K35R/ | 18 | 1.0 |
| K42R/K49Q/K50R.5d-K04Q | | |
| C-K04R/K07R/G29A/S33R/K35R/ | 20 | 0.9 |
| K42R/K49Q/K50R.5d-K07Q | | |
| C-K04R/K07R/G29A/S33R/K35R/ | 17 | 1.0 |
| K42R/K49Q/K50R.5d-K04Q/K07T | | |

Although the disclosure has been described with respect to only a limited number of embodiments, those skilled in the art, having benefit of this disclosure, will appreciate that various other embodiments may be devised without departing from the scope of the present invention. Accordingly, the scope of the present invention should be limited only by the attached claims.

What is claimed is:

1. A protein, comprising two or more amino acid sequences,
wherein each amino acid sequence is derived from a sequence selected from the group consisting of SEQ ID NOs:1 to 5,
wherein the amino acid sequence closest to the N-terminus comprises a substitution of an amino acid residue at a position corresponding to position 4 or 7 of SEQ ID NO: 5 with an amino acid residue other than Arg, and
wherein productivity of the protein in a transformant producing the protein is improved, as compared to productivity of a protein comprising the amino acid sequence without the substitution.

2. The protein according to claim 1,
wherein the amino acid residue at the position corresponding to position 4 of SEQ ID NO: 5 is substituted by Gln, Ala, Gly, Leu, Met, Phe, Pro, Val, Trp, Asn, Cys, Ser, Thr, Tyr, Asp, Glu, His, or Lys.

3. The protein according to claim 1,
wherein the amino acid residue at the position corresponding to position 7 of SEQ ID NO: 5 is substituted by Met, Phe, Asn, Gln, Thr, Tyr, His, or Lys.

4. The protein according to claim 1,
wherein, in the two or more amino acid sequences other than the amino acid sequence closest to the N-terminus, Lys is substituted by an amino acid residue other than Lys.

5. The protein according to claim 1,
wherein each amino acid sequence comprises 18 to 20 amino acid residues corresponding to amino acid residues of SEQ ID NO: 5 selected from the group consisting of Gln-9, Gln-10, Phe-13, Tyr-14, Leu-17, Pro-20, Asn-21, Leu-22, Gln-26, Arg-27, Phe-30, Ile-31, Leu-34, Pro-38, Ser-39, Leu-45, Leu-51, Asn-52, Gln-55, and Pro-57.

6. A DNA, encoding the protein according to claim 1.

7. A vector, comprising the DNA according to claim 6.

8. A transformant, produced by transforming a host cell with the vector according to claim 7.

9. A method for producing the protein according to claim 1, the method comprising:
preparing a vector comprising a DNA encoding the protein;
obtaining a transformant by transforming a host cell with the vector; and
producing the protein using the transformant.

10. A method for producing the protein according to claim 1, the method comprising:
preparing a DNA encoding the protein; and
producing the protein using a cell-free protein synthesis system comprising the DNA.

11. An affinity separation matrix, comprising:
a carrier made of a water-insoluble base material; and
an affinity ligand immobilized on the carrier, wherein the affinity ligand is the protein according to claim 1.

12. The affinity separation matrix according to claim 11,
wherein the affinity ligand binds to a protein comprising an immunoglobulin Fc region.

13. The affinity separation matrix according to claim 12,
wherein the protein comprising an immunoglobulin Fc region is an immunoglobulin G or an immunoglobulin G derivative.

14. A method for preparing the affinity separation matrix according to claim 11, the method comprising
immobilizing the protein onto the carrier.

15. A method for purifying a protein comprising an immunoglobulin Fc region, the method comprising
adsorbing a polypeptide comprising an immunoglobulin Fc region onto the affinity separation matrix according to claim 11.
